(19) United States
(12) Patent Application Publication
(10) Pub. No.: US 2024/0018393 A1
(43) Pub. Date: Jan. 18, 2024

(54) Hydrophilic Hot Melt Adhesive and Uses Thereof

(71) Applicants: Henkel AG & Co., KGaA, Dusseldorf (DE); The Procter & Gamble Company, Cincinnati, OH (US)

(72) Inventors: Jinyu Chen, Bridgewater, NJ (US); Gueltekin Erdem, Beijing (CN); Yuhong Hu, Bridgewater, NJ (US); Torsten Lindner, Schwalbach am Taunus (DE)

(21) Appl. No.: 18/451,260

(22) Filed: Aug. 17, 2023

Related U.S. Application Data
(63) Continuation of application No. PCT/CN2021/077150, filed on Feb. 22, 2021.

(51) Int. Cl. C09J 7/35; C09J 11/08; C09J 123/08

(52) U.S. Cl. CPC C09J 7/35; C09J 11/08; C09J 123/0853; C09J 2301/304; C09J 2301/312; C09J 2301/50

(57) ABSTRACT

The invention provides hydrophilic hot melt adhesive composition with improved water wetting properties. The hydrophilic hot melt adhesive is particularly suitable in the manufacture of disposable nonwoven articles.

### FIELD OF THE INVENTION

The invention relates to hydrophilic hot melt adhesive composition and uses thereof. The hydrophilic hot melt adhesive provides improved water wetting properties and are particularly suitable in the manufacture of disposable nonwoven articles.

### BACKGROUND OF THE INVENTION

Nonwovens are used commercially for a variety of applications including insulation, packaging, household wipes, surgical drapes, medical dressings, and in disposable articles such as diapers, adult incontinent products and sanitary napkins.

In many of the aforementioned applications it is necessary to adhere the nonwoven to another substrate or component. The second substrate may be another nonwoven, tissue, or an unrelated material. A commonly employed technique to bond the assembly together is the use of a hot melt adhesive. The hot melt adhesives allow for cost and time efficient manufacturing since there is no evaporation step necessary as is the case for water based or solvent based adhesive systems. Suitable hot melt adhesives must possess excellent adhesion to the substrates involved. For nonwoven applications they must also possess good flexibility, no staining or bleed through, suitable viscosity, set speed and open time to function on commercially available equipment, and acceptable thermal aging properties.

WO00/00229 (Lindner et al.) discloses hygienic articles comprising an oil resistant, hydrophilic adhesive. The formula of the adhesive or its permanency is however not disclosed. U.S. Pat. No. 6,380,292B1 (Bostik) discloses hydrophilic hot melt adhesive compositions suitable for nonwoven disposable articles which are prepared by blending various adhesive components with a surfactant.

It is desirable for the hot melt adhesive to have the ability to transmit liquid from the nonwoven substrate into the superabsorbent or fluff core substrates. This property is important in disposable diaper, sanitary napkin and bed pad constructions where it is desired to draw the moisture away from the body and into the absorbent core as quickly as possible after the nonwoven is wetted.

### BRIEF SUMMARY OF THE INVENTION

The invention provides hydrophilic hot melt adhesive composition with improved water wetting properties.

In one aspect, the invention is directed to a hydrophilic hot melt adhesive composition comprises:
(a) about 20 to about 90 wt % of an ethylene copolymer selected from the group consisting of ethylene vinyl acetate, ethylene n-butyl acrylate, ethylene octene, ethylene propylene, ethylene butene propylene, and mixtures thereof; and
(b) about 1 to about 25 wt % a nonionic, ethoxylate surfactant polymer emulsifier having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons;
   The composition exhibits a film contact angle with distilled water of less than 70 degrees both before and after aging for 15 days at 60° C.

In another aspect, the invention is directed to a method of forming a hydrophilic hot melt adhesive composition, comprising the steps of:
(a) mixing a mixture at 120° C. to about 190° C. to form a melted mixture comprising (i) about 20 to about 90 wt % of an ethylene copolymer, (ii) 1 to about 25 wt % a nonionic, ethoxylate surfactant polymer emulsifier, (iii) about 20 to about 70 wt % of a tackifying agent, (iv) up to about 20 wt % of a plasticizer, and (v) up to about 3 wt % of an antioxidant,
   and
(b) pelletizing or forming blocks of the melted mixture to solidified hydrophilic hot melt adhesive composition.

The ethylene copolymer is selected from the group consisting of ethylene copolymer selected from the group consisting of ethylene vinyl acetate, ethylene n-butyl acrylate, ethylene octene, ethylene propylene, ethylene butene propylene, and mixtures thereof. The nonionic, ethoxylate surfactant polymer emulsifier has (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons.

Yet in another aspect, the invention is directed to an article comprising the hydrophilic hot melt adhesive composition of above and a substrate comprising film, nonwoven, wood, wood pulp, cellulose, paper, cardboard, tissue, cotton, superabsorbent particles, and the like. The articles include disposable absorbent articles, mask, tissue, book, furniture, film, meat pad, animal pad, personal protective equipment, insulation, packaging, household wipes, and the like.

### DETAILED DESCRIPTION OF THE INVENTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Numerical values in the specification and claims of this application, particularly as they relate to polymers or polymer compositions, reflect average values for a composition that may contain individual polymers of different characteristics. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values. As used herein, approximating language may be applied to modify any quantitative representation that may vary without resulting in a change in the basic function to which it is related. Accordingly, a value modified by a term or terms, such as “about,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%”, and “about 1” may mean from 0.9-1.1. Other meanings of “about” may be apparent from the context, such as rounding off, so, for example “about 1” may also mean from 0.5 to 1.4.

A particularly preferable embodiment of the hygienic article of the present invention is the disposable absorbent article.

As used herein, the term “absorbent article” refers to devices which absorb and contain body exudates, and more specifically, refers to devices which are placed against the skin of the user to absorb and contain the various exudates discharged from the body of the user. Examples of disposable absorbent articles include feminine hygiene garments such as sanitary napkins and pantiliners, diapers, adult incontinence devices, diaper holders, training pants, and the like.

The term “disposable” is used herein to describe hygienic articles which are not intended to be laundered or otherwise restored or reused as a hygienic article after a single use.

Disposable absorbent articles typically comprise a liquid pervious topsheet, a liquid impervious backsheet joined to the topsheet and an absorbent core positioned between the topsheet and the backsheet.

The topsheet is a liquid pervious, permitting liquids (e.g., menses and/or urine) to readily penetrate through its thickness. A suitable topsheet may be manufactured from a wide range of materials such as woven and nonwoven materials (e.g., a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the topsheet comprises a nonwoven web, the web may be manufactured by a wide number of known techniques. For example, the web may be spunbonded, carded, wet-laid, melt-blown, hydroentangled, combinations of the above, or the like.

In general, the absorbent core is capable of absorbing or retaining liquids (e.g., menses, urine, and/or other body exudates). The absorbent core may be manufactured in a wide variety of sizes and shapes (e.g., rectangular, oval, hourglass, “T” shaped, dog bone, asymmetric, etc.). In addition to the absorbent composites of the present invention, the absorbent core may include any of a wide variety of liquid-absorbent materials commonly used in absorbent articles.

The backsheet is impervious to liquids (e.g., menses and/or urine) and is preferably manufactured from a thin plastic film, although other flexible liquid impervious materials may also be used. The backsheet prevents the exudates absorbed and contained in the absorbent core from wetting articles which contact the absorbent article such as bedsheets, pants, pajamas and undergarments. The backsheet may thus comprise a woven or nonwoven material, polymeric films such as thermoplastic films of polyethylene or polypropylene, or composite materials such as a film-coated nonwoven material. A suitable backsheet is a polyethylene film having a thickness of from about 0.012 mm (0.5 mil) to about 0.051 mm (2.0 mils). The backsheet is preferably embossed and/or matte finished to provide a more clothlike appearance. Further, the backsheet may permit vapors to escape from the absorbent core (i.e., the backsheet is breathable) while still preventing exudates from passing through the backsheet. The size of the backsheet is dictated by the size of the absorbent core and the exact absorbent article design selected.

The backsheet and the topsheet are positioned adjacent the garment surface and the body surface, respectively, of the absorbent core. The absorbent core is preferably joined with the topsheet, the backsheet, or both in any manner as is known by attachment means such as those well known in the art. However, embodiments of the present invention are envisioned wherein portions of the entire absorbent core are unattached to either the topsheet, the backsheet, or both.

For example, the backsheet and/or the topsheet may be secured to the absorbent core or to each other by a uniform continuous layer of adhesive. In order to allow efficient manufacture of the disposable absorbent articles of the present invention, hot melt adhesives are preferred for the present invention. The adhesive is in patterned layer of adhesive, or an array of separate lines, spirals, or spots of adhesive. An exemplary attachment means of an open pattern network of filaments comprises several lines of adhesive filaments swirled into a spiral patter. Alternatively, the attachment means may comprise heat bonds, pressure bonds, ultrasonic bonds, dynamic mechanical bonds, or any other suitable attachment means or combinations of these attachment means as are known in the art.

The hot melt adhesive must possess moderate to excellent adhesion to the substrates involved. Conventional hot melt adhesives are hydrophobic in nature. They typically provide a barrier between the top layer to the core and hinder hydrophilic materials, e.g., moisture, water, blood, urine, menses, to flow into the core. The present hydrophilic hot melt adhesive facilitates the transmission of the aqueous materials from the topsheet to the superabsorbent core or fluff core substrate due to its low contact angle. The hydrophilic hot melt adhesive may be used as a construction adhesive between different layers or components of the article, e.g. between the topsheet and acquisition layer, or acquisition layer and absorbent core, or between absorbent material and top core wrap layer. The hotmelt adhesives of the invention are also compatible with typical skincare lotions that may be present on the topsheet or used by a caregiver.

The hydrophilic hot melt adhesive composition comprises:
(a) about 20 to about 90 wt % of an ethylene copolymer selected from the group consisting of ethylene vinyl acetate, ethylene n-butyl acrylate, ethylene octene, ethylene propylene, ethylene butene propylene, and mixtures thereof; and
(b) about 1 to about 25 wt % a nonionic, ethoxylate surfactant polymer emulsifier having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons;

The composition exhibits a film contact angle with distilled water of less than 70 degrees both before and after aging for 15 days at 60° C.

The polymer of the hydrophilic hot melt adhesive is ethylene copolymer. The ethylene copolymer may be an ethylene vinyl acetate, ethylene n-butyl acrylate, ethylene octene, ethylene propylene, ethylene butene propylene or mixtures thereof. The ethylene monomers may have a content as low as 3 wt % of the copolymer; however, ethylene homopolymers are not a preferred polymer for the hydrophilic hot melt adhesive.

The ethylene copolymer is present in the hydrophilic adhesive composition in amounts of from about 20% to about 90%, by weight, and preferably from about 30% to about 70%.

The nonionic, ethoxylate surfactant polymer emulsifier of the hydrophilic hot melt adhesive is a solid, polymer additive and a wetting agent. It has a high molecular weight, ranging from about 400 to about 5000 Daltons, preferably from about 500 to about 3000. The melting point of the ethoxylated surfactants ranges from 70 to about 120° C., preferably from about 80 to about 100° C., more preferably from about 85 to about 95° C. The ethoxylated surfactants are fully saturated, long linear chain C20 to C50 synthetic alcohols. This long polymer chain allows for easy incorporation into the polymer system of the adhesive, and its thermostability is significantly improved. The polymer emulsifier provides bulk strength and allows for good wetting properties. The ethoxylated surfactant polymer emulsifier has a hydrophile-lipophile balance (HLB) number of less than 20, and is incorporated such that the resultant adhesive has a contact angle of 70° or less, and preferably less than about 50°.

The nonionic, ethoxylate surfactant polymer emulsifier differs from conventional nonionic ethoxylate alcohols and phenols. The conventional nonionic ethoxylate alcohols and phenols are converted into R(OC₂H₄)ₙOH, where n ranges from 1 to 10; and they are low molecular weight emulsifiers, have short chains, and have much lower melting temperatures, typically molten at ambient temperature. These conventional, low molecular weight nonionic materials include ATMER 688 from Croda, and described in WO 97/48779 and U.S. Pat. No. 6,380,292 fail to provide low contact angles. Therefore, conventional nonionic ethoxylate alcohols and phenols are very mobile and cannot be stabilized in a polymer matrix. As a result, they will easily migrate to the surface of a polymer matrix and phase separate from the matrix, even at room temperature. Furthermore, given their motility in the system, their capability to decrease the contact angle is unstable and short-lived. On the contrary, the addition of a nonionic, ethoxylate surfactant polymer emulsifier that have high molecular weight, long chain and high melting temperature, provides stability in the polymer matrix, and as a result, the contact angle reduction is consistent and durable, as evidenced in the unchanged contact angle after accelerated ageing tests.

The nonionic, ethoxylate surfactant polymer emulsifier can be present in the hydrophilic adhesive composition in amounts of from about 1% to about 25%, by weight, and preferably from about 1% to about 10%.

The ethoxylated surfactant must be reasonably compatible with the other raw materials used in the hot melt adhesive so that it does not adversely affect the construction performance of the adhesive. On the other hand, the ethoxylated surfactant must “bloom” to the surface of the adhesive so as to lower the contact angle and make the adhesive more hydrophilic. Thus, a delicate balance of compatibility must be maintained.

Optional components may be further added to the hydrophilic adhesive, such as tackifier, plasticizer, antioxidant, and the like.

The tackifying resins useful in the adhesive compositions can be hydrocarbon resins, synthetic polyterpenes, rosin esters, natural terpenes, and the like. More particularly, and depending upon the particular base polymer, the useful tackifying resins may include any compatible resins or mixtures thereof such as (1) natural and modified rosins such, for example, as gum rosin, wood rosin, talloil rosin, distilled rosin, hydrogenated rosin, dimerized rosin, and polymerized rosin; (2) glycerol and pentaerythritol esters of natural and modified rosins, such, for example as the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; (3) copolymers and terpolymers of natured terpenes, e.g., styrene/terpene and alpha methyl styrene/terpene; (4) polyterpene resins having a softening point, as determined by ASTM method E28-58T, of from about 80° to 150° C.; the latter polyterpene resins generally resulting from the polymerization of terpene hydrocarbons, such as the bicyclic monoterpene known as pinene, in the presence of Friedel-Crafts catalysts at moderately low temperatures; also included are the hydrogenated polyterpene resins; (5) phenolic modified terpene resins and hydrogenated derivatives thereof such, for example, as the resin product resulting from the condensation, in an acidic medium, of a bicyclic terpene and a phenol; (6) aliphatic petroleum hydrocarbon resins having a Ball and Ring softening point of from about 70° to 135° C.; the latter resins resulting from the polymerization of monomers consisting of primarily of olefins and diolefins; also included are the hydrogenated aliphatic petroleum hydrocarbon resins; (7) aromatic petroleum hydrocarbon resins and the hydrogenated derivatives thereof; and (8) alicyclic petroleum hydrocarbon resins and the hydrogenated derivatives thereof. Mixtures of two or more of the above described tackifying resins may be required for some formulations.

While the tackifier may comprise up to about 70% of the adhesive, it is generally used in amounts of about 20 to 50% by weight.

A plasticizer can be present in the composition of the present invention in amounts of up to about 20% by weight, preferably up to about 10 wt %, based on the total weight of the adhesive, in order to provide desired viscosity control without substantially decreasing the adhesive strength, the service temperature of the adhesive, and hydrophilicity of the adhesive. A suitable plasticizer may be selected from the group which not only includes the usual plasticizing oils, such as mineral oil, naphthenic, paraffinic, Gas to Liquid (GTL) oil, but also olefin oligomers and low molecular weight polymers, as well as vegetable and animal oil and derivatives of such oils. The petroleum derived oils which may be employed are relatively high boiling temperature materials containing only a minor proportion of aromatic hydrocarbons. In this regard, the aromatic hydrocarbons should preferably be less than 30%, and more particularly less than 15%, by weight, of the oil. Alternately, the oil may be totally non-aromatic. The oligomers may be polypropylenes, polybutenes, hydrogenated polyisoprene, hydrogenated butadiene, or the like having average molecular weights between about 350 and about 10,000. Suitable vegetable and animal oils include glycerol esters of the usual fatty acids and polymerization products thereof. The plasticizer that finds usefulness in the present invention can be any number of different plasticizers, but the inventors have discovered that mineral oil such as Kaydol manufactured by Witco, is particularly useful in the present invention. Benzoflex 9-88, a dipropylene glycol dibenzoate manufactured by Velsicol, has also been found to be an appropriate plasticizer. Risalla X 409, a Gas to Liquid oil from Shell, is also suitable for a plasticizer. As will be appreciated, plasticizers have typically been employed to lower the viscosity of the overall adhesive composition without substantially decreasing the adhesive strength and/or the service temperature of the adhesive. The choice of plasticizer can be useful in formulation for specific end uses (such as wet strength core applications).

Among the applicable stabilizers or antioxidants which may be included herein are high molecular weight hindered phenols and multifunctional phenols such as sulfur and phosphorous-containing phenols. Representative hindered phenols include: 1,3,5-trimethyl-2,4,6-tris(3,5-di-tert-butyl-4-hydroxyphenyl) propionate; n-octadecyl 3,5-di-tert-butyl-4-hydroxyphenyl) propionate; 4,4′-methlenebis (2,6-di-tert-butylphenol); 4,4′-thiobis (6-tert-butyl-o-cresol); 2,6-di-tert-butylphenol; 6-(4-hydroxyphenoxy)-2,4-bis(n-octylthio)-1,3,5-triazine; di-n-octadecyl-3,5-di-tert-butyl-4-hydroxy-benzylphosphonate; 2-(n-octylthio)-ethyl 3,5-di-tert-butyl-4-hydroxybenzoate; and sorbitol hexa[3-(3,5-di-tert-butyl-4-hydroxyphenyl)propionate]. If used, the stabilizer is present in levels of about 0.1 to 3% by weight.

Optional additives may be incorporated into the hot melt compositions in order to modify certain properties thereof. Among these additives may be included wax, colorants such as titanium dioxide; and fillers such as talc and clay, etc.

A low contact angle is desirable so that water, urine or other water-based discharges “wet out” rather than “bead up” resulting in the fluid being directed into the core and away from the topsheet. The hydrophilicity of an adhesive can be quantified by the Adhesive Contact Angle Measurement described hereinafter. In order to be suitable for the absorbent article of the present invention, the adhesive must have a contact angle with distilled water of less than 70°, more preferably less than 50° C.

The hydrophilic hot melt adhesive compositions of the invention may be formulated using techniques known in the art. An exemplary procedure involves placing all the polymer, surfactant, tackifiers, plasticizers and stabilizers in a jacketed mixing kettle, preferably in a jacketed heavy duty mixer, which is equipped with rotors and thereupon raising the temperature to a range of from about 120° C. to up to about 190° C. After the resin has melted, the temperature is lowered to 100° to 165° C. Mixing and heating are continued until a smooth, homogeneous mass is obtained.

The hydrophilic hot melt adhesive may be applied onto various substrates including film, nonwoven, cellulose or synthetic fibers, or superabsorbent particles. The hydrophilic hot melt adhesive may be in particular used to bond a first component which is a nonwoven to a second component of the absorbent articles, which may be a second nonwoven, or cellulose fibers, or superabsorbent particles, or a combination thereof. The hydrophilic hot melt adhesive facilitates the transfer of liquid such as urine from one component to the next component thanks to its hydrophilicity through the use of the article, which may be several hours and cover several liquid insults. The hydrophilic hot melt adhesive may be used for example to attach the topsheet to an acquisition layer, an acquisition layer to a distribution layer, an acquisition or a distribution layer to the absorbent core, a topsheet directly or indirectly to the absorbent core, the core wrap, in particular the inner surface of the top side of the core wrap, may also be attached to the absorbent layer by the hot melt adhesive of the invention to facilitate the transfer of liquid from the surface of the core wrap into the absorbent layer. The absorbent layer may be a mixture of cellulose fibers and superabsorbent particles, or superabsorbent particles substantially free of cellulose fibers. The absorbent layer, in particular for absorbent layer consisting of superabsorbent particles without cellulose fibers, may also be immobilized within the core wrap by a hot melt adhesive according to the invention.

The hydrophilic hot melt adhesive composition may be applied onto various substrates including, film, nonwoven, wood, wood pulp, cellulose, paper, cardboard, tissue, cotton, superabsorbent particles, and the like.

As noted above, the resulting adhesives may be employed in a wide variety of uses as are known in the art. In particular, adhesives are useful for the assembly of disposable articles using multi-line, spray, or slot-coating construction techniques wherein at least one liquid pervious substrate is bonded to at least one tissue, non-woven, polyolefin or other flexible polymeric film substrate. In addition, the adhesives may be useful in bonding elastic to polyethylene, polypropylene or non-woven substrate to impart elongation resistant gathers thereto. When formulated with plasticizers, the resultant adhesives may be used in the assembly or construction of various disposable applications including, but not limited to, sanitary napkins, disposable diapers, hospital gowns, bed pads and the like. The adhesive may also be utilized in less demanding disposable construction applications such as for end or perimeter sealing. They may also be used for laminating or coating tissue and/or screen-reinforced tissue layers such as are used in individual or roll use applications as in wipes, tissues, labels, paper towels, toilet tissue, household wipes, personal protective equipment, masks, meat pads, animal pads, furniture, and other consumer or industrial end uses, e.g., film, insulation. The adhesives may be effectively utilized in a variety of packaging and carton sealing applications. The adhesives may also be used to bind a plurality of sheets in a wide range of bookbinding operations and in insulation and furniture.

In addition, the resulting adhesive could also be used as a hydrophilic coating to reduce contact angle of the substrates and to enhance hydrophilicity of the substrate surfaces. This hydrophilic coating has many applications. The coating can be applied as anti-fog coating to any surface to flatten water drops and to prevent bead formation. In another example, the coating is applied onto substrates that comes in contact with biological systems, particularly biological fluids. Hydrogen in the biological fluids bonds to the hydrogen bonding sites of the hydrophilic coating. In addition, the coating is useful as anti-graffiti coating since it can resist oil adsorption. Moreover, hydrophilic coating can be applied on adhesive film as polymeric electrolytes in battery and ion conduction applications.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### Examples

Viscosity: Viscosity was measured with a Brookfield viscometer, spindle #27 at 150° C., in accordance with ASTM 3236-88.

Contact Angle Test: As a drop of liquid meets a solid surface, it assumes a distinctive shape. The shape and length of time that it holds onto its shape are determined by three interfacial tension forces: the force of the solid surface, the surface tension of the liquid and the force at the solid/liquid interface. The contact angle (θ) is a measured value relative to the combined vector forces according to the formula:

γ_{L} COS θ=γ_{S}−γ_{SL}

where γ_{L} is the interfacial tension of the liquid/air boundary, γ_{S} is the interfacial tension of the solid/air boundary, γ_{SL} is the interfacial tension of the solid/liquid boundary, and θ is the angle of the liquid drop.

The goniometer has a microsyringe for dispersing accurate droplet sizes and a camera for photographing the angle of the liquid drop as it meets the surface of the solid. The contact angle is measured as the angle between the substrate and the tangent of the liquid drop (at the interface).

The lower the angle, the more effective the coating is in transmitting the liquid through the adhesive layer. The contact angle for Fresh and Aged film sample is measured as follows:

Fresh sample: a 50 micrometer thick film of adhesive coating is made on a PET film by a hot melt coater laminator (e.g. HLCL-1000, ChemInstruments) at 120° C. (adapt temperature if needed) and then let it cool down to ambient lab conditions (21-23° C., 40%-60% RH). The contact angle testing is conducted on the adhesive surface 24 hour after the adhesive coating making; and.

Aged sample: the fresh sample is put into a convection oven (e.g. Blue M convection oven, Stabil-Therm) and subjected to 15 days of aging. The oven temperature is set to 60° C. After ageing, the sample is taken out from the oven and conditioned at ambient lab conditions (21-23° C., 40%-60% RH) for 24 hours before the contact angle on adhesive surface is measured.

Instron Testing: The bond strength was evaluated on Instron Tester. The laminate bonded with hot melt adhesive was separated at the ends and placed in tensile tester jaws. The sample was then pulled at 12 in/min crosshead speed, and the average peel value recorded in grams or pounds for each product tested. If there was bond failure, the type of failure was recorded instead of peel value.

Table 1 lists the ethoxylate surfactant and their properties used in the examples.

TABLE 1

| | | | | Hydroxyl | |
|---|---|---|---|---|---|
| | | Ethylene | | number | |
| | Calculated | oxide (% by | | (mg KOH/g | Melting point |
| Ethoxylated | Mw | weight) - NMR | | sample) - | (° C.)-ASTM |
| surfactant | (Daltons) | calculation | HLB value | ASTM #-222 | D-127 |
| Unithox 420* | 575 | 20 | 4 | 85 | 91 |
| Unithos 450* | 920 | 50 | 10 | 55 | 91 |
| Unithox 480* | 2300 | 80 | 16 | 22 | 86 |

*Baker Hughes

Table 2 lists all the polymers used in the examples and their properties.

TABLE 2

| | | | | Ring& Ball | |
|---|---|---|---|---|---|
| | Polymer | Ethylene | Melt Flow | Softening | Tm |
| Polymer Name | Type | content (%) | (g/10 min)* | Point (° C.)** | (° C.) *** |
| Ateva¹ 3342 | EVA | 67 | 400 | 81 | |
| Ateva¹ 4030 | EVA | 60 | 55 | 107 | 52 |
| Ateva¹ 2842 | EVA | 72 | 400 | 84 | 65 |
| Ateva¹ 1850 | EVA | 82 | 150 | 95 | 84 |
| Enable² 33331 | EnBA | 67.5 | 330 | | 62 |
| Affinity³1875 | PE-Octene | 65 | 1250 | | 70 |
| Vistamaxx²8780 | PP-E | 12 | Viscosity | | 96 |
| | | | 3980 cps at | | |
| | | | 190° C. | | |
| Vestplast⁴ 508 | PP/B/E | 3 | Viscosity | 84 | |
| | | | 8000 cps at | | |
| | | | 190° C. | | |
| RT 2830⁵ | PP-B | 0 | Viscosity | 90 | |
| | | | 3000 cps at | | |
| | | | 190° C. | | |

*ASTM D 1238 @ load 2.16 kg @ 190° C.
**ASTM E 28
*** ASTM D 3418
¹Celanese
²ExxonMobile
³Dow
⁴Evonik
⁵Rextac

Table 3 demonstrated the contact angle of each formulation with various polymers.

The representative tackifier used in the example is Escorez 5690, an aromatic modified cycloaliphatic hydrocarbon resins with softening point of 90.5° C. from ExxonMobil.

The representative plasticizer used in the examples is Calsol 5550, a naphthenic oil from Calumet.

The representative antioxidant used in the examples is Evernox 10 from Everspring.

Film contact angles measured 24 hours after making the samples (“Fresh” samples) and aged for 15 days at 60° C. (“Aged” samples) are shown in Table 3. Film contact angle with distilled water of less than 70 degrees both before and after aging are considered as pass (P). Contact angles greater than 70 degrees is considered as fail (F), as described in the comments.

TABLE 3

| Example | 1 | 2 | 3 | 4 | 5 | 6 | 7 | 8 | 9 | 10 |
|---|---|---|---|---|---|---|---|---|---|---|
| Escorez | | | | 45 | 45 | 45 | 45 | 45 | 45 | 45 |
| 5690 | | | | | | | | | | |
| Unithox | | 5 | 10 | | 18 | 18 | 18 | 18 | 18 | 18 |
| 450 | | | | | | | | | | |
| Ateva | 100 | 95 | 90 | 54 | 36 | | | | | |
| 2842 | | | | | | | | | | |
| Enable | | | | | | 36 | | | | |
| 33331 | | | | | | | | | | |
| Vistamaxx | | | | | | | 36 | | | |
| 8780 | | | | | | | | | | |
| RT 2830 | | | | | | | | 36 | | |
| Vestoplast | | | | | | | | | 36 | |
| 508 | | | | | | | | | | |
| Affinity | | | | | | | | | | 36 |
| 1875 | | | | | | | | | | |
| Evernox | | 1 | 1 | 1 | 1 | 1 | 1 | 1 | 1 | 1 |
| 10 | | | | | | | | | | |
| Viscosity | | 44150 | 30000 | 10150 | 2000 | 2375 | 470 | 1220 | 1950 | 575 |
| @ 150° C. | | | | | | | | | | |
| (cps) | | | | | | | | | | |
| Contact | 82 | 37 | 39 | 91 | 31 | 30 | 30 | 74 | 41 | 23 |
| Angle- | | | | | | | | | | |
| Fresh (°) | | | | | | | | | | |
| Contact | 87 | 34 | 38 | 91 | 33 | 27 | 40 | 87 | 35 | 30 |
| Angle- | | | | | | | | | | |
| 15 days at | | | | | | | | | | |
| 60° C.- | | | | | | | | | | |
| Aged (°) | | | | | | | | | | |
| Comment | F | P, but | P | F | P | P | P | F | P | P |
| | | high | | | | | | | | |
| | | viscosity | | | | | | | | |

The pure polymers all have relatively high contact angle, greater than 80° for example: Ateva 2842 alone, Example 1, has a contact angle of 82° at fresh and 87° after aging at 160° C. for 15 days. Without the nonionic ethoxylate surfactant polymer emulsifier, the contact angle remains high, Example 4. When this polymer is mixed with ethoxylate surfactant, Example 2, its thin film contact angle is substantially reduced to 37° for Fresh sample, and 34° after aging at high temperature (Aged).

The ethylene-based polymer has a good compatibility with the nonionic ethoxylate surfactant polymer emulsifier, leading to a homogeneous system that is critical for the stable wetting properties of end adhesive. As seen, both Fresh and Aged thin film maintain low contact angle (<70°) for EVA (Examples 2, 3, 5), EnBA (Example 6), PE-Octene (Example 10), PP-E (Example 7), and PP/B/E (Example 9), but fail on PP-B (Example 8). PP-B is not compatible with Unithox 450 due to poor miscibility of each other.

Table 4 shows the formulation using EVA with different VA content together with Unithox 450 and Escorez 5690. As seen, while different types of EVA may tune the viscosity range, they do not substantially impact the contact angles of the system, all below 70° for both Fresh and Aged conditions.

TABLE 4

| Example | 11 | 12 | 13 | 14 |
|---|---|---|---|---|
| Escorez 5690 | 45 | 45 | 45 | 45 |
| Unithox 450 | 5 | 5 | 5 | 5 |
| Ateva 1850A, 18% VA content | 49 | | | |
| Ateva 2842A, 28% VA content | | 49 | | |
| Ateva 3342, 33% VA content | | | 49 | |
| Ateva 4030, 40% VA content | | | | 49 |
| Evernox 10 | 1 | 1 | 1 | 1 |
| Viscosity @ 150° C. (cps) | 14,400 | 6,725 | 6,700 | 33,500 |
| Contact Angle - Fresh (°) | 29 | 37 | 37 | 42 |
| Contact Angle - 15 days at | 31 | 34 | 47 | 47 |
| 60° C. Aged (°) | | | | |
| Comments | Pass | Pass | Pass | Pass |
| | | | | but high |
| | | | | viscosity |

Table 5 shows the impact of the ethoxylate surfactant polymer emulsifier on the contact angle. Unithox 420,450 and 480 all result in very low contact angle of hot melt adhesives.

TABLE 5

| Example | 5 | 15 | 16 |
|---|---|---|---|
| Escorez 5690 | 45 | 45 | 45 |
| Unithox 420 | | | 9 |
| Unithox 450 | 18 | | |
| Unithox 480 | | 18 | 9 |
| Ateva 2842A | 36 | 36 | 36 |
| Evernox 10 | 1 | 1 | 1 |
| Viscosity @ 150° C. (cps) | 2000 | 2500 | 2300 |
| Contact Angle - Fresh (°) | 31 | 34 | 24 |
| Contact Angle - 15 days at 60° C. Aged (°) | 33 | 32 | 48 |
| Comments | Pass | Pass | Pass |

Table 6 shows a formulation with plasticizers (Examples 17-19). The addition of plasticizer provides better wetting properties. Ideally, the total content of the plasticizer is less than 20 wt % of the adhesive.

TABLE 6

| Example | 4 | 5 | 17 | 18 | 19 |
|---|---|---|---|---|---|
| Escorez 5690 | 45 | 45 | 40 | 35 | 30 |
| Unithox 450 | | 18 | 5 | 5 | 5 |
| Ateva 2842 | 54 | 36 | 49 | 49 | 49 |
| Calsol 5550 | | | 5 | 10 | 15 |
| Evernox 10 | 1 | 1 | 1 | 1 | 1 |
| Viscosity @ 150° C. (cps) | 10150 | 2000 | 4780 | 4280 | 3850 |
| Contact Angle - Fresh (°) | 91 | 31 | 30 | 31 | 31 |
| Contact Angle- 15 days at | 91 | 33 | 41 | 50 | 54 |
| 60° C. (°) | | | | | |
| Comments | Fail | Pass | Pass | Pass | Pass |

Table 7 shows the bond strength of laminate made by slot coating or spiral coating adhesives onto a general-purpose top sheet nonwoven then bonding to a core nonwoven. The coat weight is 8 grams per square meter, line speed 300 meter per min. The bond strength may be optimized by adjusting the content of polymer and surfactant.

TABLE 7

| Example | 5 | 12 |
|---|---|---|
| Bond strength by slot coated (gram/in) | 58 | 206 |
| Bond strength by spiral coated(gram/in) | 51 | 90 |

Examples 20, 21, and 22 of Table 8 show the effect of conventional, low molecular weight, nonionic surfactant blend (ATMER 688 from Croda International) as described in WO 97/48779 and U.S. Pat. No. 6,380,292B1. The film contact angles of Examples 20, 21, and 22, are all greater than 70° at both fresh and after aging conditions. In contrast, Examples 9 and 13, made with an ethoxylate surfactant polymer emulsifier, Unithox 450, have a fresh contact angle below 70°.

TABLE 8

| Example | 9 | 13 | 20 | 21 | 22 |
|---|---|---|---|---|---|
| Escorez 5690 | 45 | 45 | 45 | 45 | 45 |
| Unithox 450 | 18 | 5 | | | |
| Atmer 688 | | | 5 | 5 | 18 |
| Vestoplast 508 | 36 | | | 49 | 36 |
| Ateva 3342, 33% VA content | | 49 | 49 | | |
| Evernox 10 | 1 | 1 | 1 | 1 | 1 |
| Viscosity @ 150° C. (cps) | 1950 | 6700 | 6600 | 5400 | 1160 |
| Contact Angle - Fresh (°) | 41 | 37 | 72 | 98 | 95 |
| Contact Angle- 15 days at 60° C. (°) | 35 | 47 | 80 | 98 | 97 |
| Comments | Pass | Pass | Fail | Fail | Fail |

We claim:

1. A hydrophilic hot melt adhesive composition comprising:
(a) About 20 to about 90 wt % of an ethylene copolymer selected from the group consisting of ethylene vinyl acetate, ethylene n-butyl acrylate, ethylene octene, ethylene propylene, ethylene butene propylene, and mixtures thereof; and
(b) About 1 to about 25 wt % a nonionic, ethoxylate surfactant polymer emulsifier having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons;
wherein the composition exhibits a film contact angle with distilled water of less than 70 degrees both before and after aging for 15 days at 60° C.

2. The hydrophilic hot melt adhesive composition of claim 1, wherein
(a) about 30 to about 70 wt % of the ethylene copolymer; and
(b) up to about 20 wt % of the ethoxylated surfactant;
wherein the composition exhibits a film contact angle with distilled water of less than 50 degrees.

3. The hydrophilic hot melt adhesive composition of claim 1, wherein the ethylene copolymer is ethylene vinyl acetate.

4. The hydrophilic hot melt adhesive composition of claim 1, further comprising:
(c) about 20 to about 70 wt % of a tackifying agent,
(d) up to about 20 w % of a plasticizer; and
(e) up to about 3 wt % of an antioxidant.

5. The hydrophilic hot melt adhesive composition of claim 4, wherein the tackifying agent is a hydrocarbon resin, a rosin ester resin, or terpene resin.

6. The hydrophilic hot melt adhesive composition of claim 5, wherein the hydrocarbon resin is selected from the group consisting of C5 aliphatic resins, C9 aromatic resins, dicyclopentadiene resins, C5/C9 aliphatic/aromatic resins, and mixtures thereof.

7. The hydrophilic hot melt adhesive composition of claim 5, wherein the rosin ester resin is rosin acids, rosin esters, hydrogenated rosin resins, dimerized rosin resins, modified rosin resins, and mixtures thereof.

8. The hydrophilic hot melt adhesive composition of claim 4, wherein the plasticizer is selected from the group consisting of naphthenic oil, paraffinic oil, vegetable oil, animal oil, olefin oligomers, and derivatives thereof.

9. A method of making a hydrophilic hot melt adhesive composition, comprising the steps of:
(a) Mixing a mixture at 120° C. to about 190° C. to form a melted mixture comprising (i) about 20 to about 90 wt % of an ethylene copolymer, (ii) 1 to about 25 wt % a nonionic, ethoxylate surfactant polymer emulsifier, (iii) about 20 to about 70 wt % of a tackifying agent, (iv) up to about 20 wt % of a plasticizer, and (v) up to about 3 wt % of an antioxidant, and
(b) Pelletizing or forming blocks of the melted mixture to solidified hydrophilic hot melt adhesive composition.

10. The method of claim 9, wherein
(a) the ethylene copolymer is selected from the group consisting of ethylene copolymer selected from the group consisting of ethylene vinyl acetate, ethylene n-butyl acrylate, ethylene octene, ethylene propylene, ethylene butene propylene, and mixtures thereof; and
(b) the nonionic, ethoxylate surfactant polymer emulsifier has (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons.

11. An article comprising the hydrophilic hot melt adhesive composition of claim 1, and a substrate comprising a film, nonwoven, wood, wood pulp, cellulose, paper, cardboard, tissue or cotton.

12. The article of claim 11, which is a mask, tissue, book, furniture, film, meat pad, animal pad, personal protective equipment, insulation, packaging, household wipes.
